(19) United States
(12) Patent Application Publication
(10) Pub. No.: US 2016/0331436 A1
(43) Pub. Date: Nov. 17, 2016

(54) PLASMA DEVICE

(71) Applicant: LINDE AKTIENGESELLSCHAFT, Munich (DE)

(72) Inventor: Thomas Bickford Holbeche, Church Crookham (GB)

(21) Appl. No.: 15/112,441

(22) PCT Filed: Jan. 22, 2015
(86) PCT No.: PCT/GB2015/000014

§ 371 Date: Jul. 19, 2016

(30) Foreign Application Priority Data
Jan. 23, 2014 (GB) 1401144.9

(51) Int. Cl. A61B 18/04; A61B 18/20

(52) U.S. Cl. CPC A61B 18/042; A61B 18/203; A61B 2018/00452

(57) ABSTRACT

A plasma-generation device for applying plasma to a human body, the device having a reservoir containing a gas, a plasma zone in fluid connection with the reservoir, means for generating a plasma by electrical discharge in the plasma zone, and a first laser generating device. The plasma zone has an outlet for dispensing plasma and the first laser generating device is arranged to generate a laser for simultaneous plasma and laser treatment of a surface.

The present disclosure relates to a non-thermal plasma treatment device and method. In particular, the disclosure relates to a device for the treatment of Dermatomycosis and other skin and nail disorders.

Recently a number of proposals have been put forward to provide a system for the generation of non-thermal (also known as non-equilibrium) gas plasma in the industrial, dental, medical, cosmetic and veterinary fields. Non-thermal gas plasma generation can be employed to promote coagulation of blood, cleaning, sterilisation, removal of contaminants from a surface, disinfection, re-connection of tissue and treatment of tissue disorders without causing significant thermal tissue damage. The plasma itself may be applied to a surface to be treated or may act as a precursor for generating a modified gaseous species that is applied to the surface.

US2003/144654 relates to a device for electrosurgery which relies upon the use of an electrical discharge. The generation of this electrical discharge has a cauterising and/or cutting effect. The discharge is created between an electrode (32) and a surface of the patient to be treated; the patient is grounded with a grounding plate (34). The device further includes a laser (25) which is used to energise gaseous atoms in the surgery site. This creates a path of least resistance for the electrosurgical arc and allows for more accurate targeting of the cutting and/or cauterising. However, there is no disclosure of using a flow of plasma and a laser to simultaneously treat a treatment surface.

U.S. Pat. No. 6,387,088 relates to a method for the controlled ablation of a surface of the human body. This apparatus relies upon the use of a UV energy source to create an irradiating beam through a selected neutral gas environment. The UV energy source causes

the generation of plasma which has the ablating effect. The generation of the plasma is aided by the presence of an electrical field between the UV device and the surface to be ablated. There is no disclosure of a flow of plasma used to simultaneously treat the same portion of the body.

It is an object of the present invention to provide an improved device and, in particular, an improved approach for the treatment of skin and nail disorders, and to tackle the drawbacks associated with the prior art, or at least provide a commercially viable alternative thereto.

According to a first aspect, there is provided a plasma-generation device for applying plasma to a human body, the device comprising:

a reservoir containing a gas,

a plasma zone in fluid connection with the reservoir,

means for generating a plasma by electrical discharge in the plasma zone, and a first laser generating device,

wherein the plasma zone has an outlet for dispensing plasma and wherein the first laser generating device is arranged to generate a laser for simultaneous plasma and laser treatment of a surface.

The present disclosure will now be described further. In the following passages different aspects/embodiments of the disclosure are defined in more detail. Each aspect/embodiment so defined may be combined with any other aspect/embodiment or aspects/embodiments unless clearly indicated to the contrary. In particular, any feature indicated as being preferred or advantageous may be combined with any other feature or features indicated as being preferred or advantageous.

The present invention relates to a plasma-generation device. That is, the device is designed to produce a plasma from the ionisation of a gas. The device is especially for producing a non-thermal plasma, as discussed herein. The plasma produced preferably has a temperature of less than 50° C, more preferably less than 45° C and most preferably from 37 to 42° C. It will be appreciated that for certain treatments, especially for hair treatment, temperature may suitably be at even higher temperatures. The device is suitable for applying plasma to a human body, which applies a number of constraints since thermal plasma production devices are clearly unsuitable. Furthermore, the production levels of UV, electrical stimulation and active species must be at levels which do not cause undue harm to a patient.

The device described herein is preferably hand-held. By hand-held, it is meant that at least the treatment application head is sized and configured such that it can be readily manipulated and controlled with one hand. Examples of hand-held devices include hair-brushes, hair-driers, toot-spa, hair-tongs, toothbrushes and the like. The treatment application head may be tethered to a power supply and/or a gas reservoir,

The ideal form for home use by a consumer is an entirely self-contained hand held device. This would have an internal battery as a power source and rely upon interchangeable gas canisters which can be clipped into the device. Nonetheless, for reasons of power requirements, it may be easier to have a mains power lead attached to the device.

Especially when the device is to be used by a professional, such as in a hair or nail salon, or by a doctor, podiatrist, or the like, it may be easier to have the hand-held device tethered to a power supply and a larger gas tank. This makes it easier for the professional to use since they do not need to change the gas tank/cartridge/camster often.

Preferably the power supply comprises a battery integrated into the hand-held device. That is, preferably the plasma-generation device is entirely independent and does not require a tether to a power supply. This increases the utility of the device in-so-far as it can be more accurately applied and can be used in a wider range of environments, such as bathrooms.

The use of a hand-held device as discussed herein has a large number of advantages. The provision of the plasma keeps the device sterile and it can be readily reused for multiple patients, in addition, the plasma produces a ready supply of active gas species which provide the treatments discussed herein. The active gas species are further supplemented by the temperature, UV light and electrical stimulation which are associated with the plasma production process.

The plasma treatment device comprises a reservoir containing gas. The reservoir acts as a source of gas from which a plasma is generated. The reservoir contains a source of pressurised gas which can be supplied to the plasma zone as the treatment application portion of the device. The gas will typically be stored in a tank (up to approximately 200 L) for professional use, or in replaceable and/or rechargeable canisters of cartridges for home use. The use, design and requirements for such sources of gas are well known in the art.

The gas is preferably Helium, Argon, Neon, Krypton, or Hydrogen, or mixtures of two or more thereof.

The reservoir is in fluid communication with a plasma zone within which plasma is created for use in a treatment. The plasma zone includes means for generating a plasma by electrical discharge therein. The plasma zone has an outlet through which the plasma passes to treat a surface. The outlet is preferably a nozzle. The plasma is conveyed by the momentum of the gas entering the plasma zone.

The device comprises a means for generating a plasma by electrical discharge through the gas. Preferably, the means for generating a plasma comprises a power supply, and first and second electrodes, and wherein, in use, the plasma zone is formed between the first and second electrodes and wherein a flow of gas from the reservoir through the plasma zone provides a flow of plasma to contact a surface of a human body. The provision of a high voltage drop between the two electrodes will cause the production of a plasma by ionising the gas provided. In this embodiment the gas flow will typically be greater so that the plasma flows out from between the electrodes and can be applied to a treatment area.

Preferably the means for generating a plasma operates at a voltage of from 2-15 kV, preferably from 3 to 10 kV and most preferably about 5 kV. These levels of voltage can he achieved in a hand-held device and still produce a suitable level of plasma generation. The power range of the device is preferably 1-100 Watts AC at a high frequency of 10-60 KHz. Alternatively, power may be delivered as high frequency pulsed DC fast rise time square waveforms.

Preferably the gas is supplied through the means for generating a plasma at a flow rate of less than 5 l/min, preferably less than 2.5 l/min, more preferably less than 1.5 l/min, preferably from 0. 1 to 1 l/min, preferably from 0.01 to 0.5 l/min. The gas flow rate for area treatments as discussed above will typically be lower than required for point treatments which require the production of a targeted jet of plasma. The flow rates for treatments which produce a plasma between a dielectric electrode a treatment are of a patient are preferably from 0.01 to 0.1 l/min. The flow rates for treatments which produce a plasma between two electrodes and rely on the gas flow to carry the plasma to a treatment are preferably from 0.5to 2.5 l/min.

Preferably the device takes the form of a hair straightener, a toothbrush, a foot-spa or a hair-brash. In these recognisable forms, the consumer is already familiar with the usage requirements and application techniques required to employ the device. This avoids any hurdle to application. More particularly, these application devices are suitable for the application of the plasma to the regions that specifically require treatment, such as the hair or teeth of a user. The device may be a hand-piece for use by a podiatrist or a patient.

The device, further comprises a first laser generating device, and optionally a second laser generating device. Lasers are well known in the art. The laser generating device or devices are arranged to generate a laser for simultaneous plasma and laser treatment of a surface. Preferably the laser has an operating frequency with a wavelength in the range of from 10 to 635 nm, more preferably 405 to 635 nm. This is particularly suitable for treating various skin complaints, as discussed herein. When the device includes first and second lasers, the wavelength of each laser may be independently selected. Preferably the first laser generating device is arranged to generate a laser that is collinear with a longitudinal axis of the nozzle.

The inventors have found that they can combine a laser treatment with a plasma treatment to provide high efficacy treatment of certain skin disorders. Moreover, they have provided a specific design of treatment device which allows the combined focusing of the laser and the plasma on the same treatment sites.

Advantageously, the inventors have found that the laser can provide a complementary treatment to the plasma treatment. For example plasma used to kill Trichophyton rubrum, the pathogen responsible for dermatophytic onychomycosis, which can simultaneously be treated with laser light in the frequency range 635 nm to 405 nm. Thus, a single device provides a means of delivering both treatments together simultaneously. A Podiatrist would be able to use the device to augment the known laser treatment of Onychomycosis with plasma and be able to direct the treatment locally and accurately so improving the effectiveness of both treatments.

The device could also be used in the dental field for dental hygiene, destroying bacteria in the mouth and treating halitosis. In particular, a potential use in the dental field is where laser UV spectrum light (10 to 400 nm, preferably 300 to 350 nm) is used to cure polymer materials used in fillings and crowns that are closer to the natural tooth colour than conventional Mercury amalgam. The device could allow the dental practitioner to not only cure a crown or filling material by selecting the appropriate frequency laser to which the filling polymer polymerises at, but also use the plasma jet to activate the tooth substrate to form a better bond.

Moreover, the anti-bacterial effects of atmospheric cold plasma are well established and this could also be of further benefit in a combined device as the dentist could sterilise the cavity or tooth surface with the plasma jet prior to starting to build the filling. This would mean that the dentist would have the advantage of a tool capable of a number of different applications in one hand piece.

The device allows for simultaneous plasma and laser treatment of a surface. It will be appreciated that the device may in one embodiment involve the switching between plasma and laser treatment. That is, the treatment may be sequential and alternating between the laser and plasma on the treatment surface. Such alternating treatment would preferably be rapidly alternating such as 1 -60 Hz. Preferably the laser and fee plasma are applied such that the treatment times overlap and are at least simultaneous for a portion of the treatment.

Preferably the device further comprises a second laser generating device, and wherein the first and second laser generating devices are arranged to generate lasers which intersect at a predefined spacing from the outlet. This is shown in FIG. 2. Advantageously, this configuration allows for the plume of the plasma to be accurately held at a desirable spacing from the surface to be treated. Thus, the plasma treatment can, for example, be optimised for the intensity of treatment best suited for a given skin disorder. This is particularly advantageous because the plasma plume can be quite faint and the operator could struggle to arrange the device accurately for a treatment. Moreover, the use of two separate lasers permits the use of two different wavelengths of light used to treat the treatment area. This could allow, for example, one wavelength intended to stimulate blood flow and another intended to kill fungus.

When using two lasers, one or neither of the lasers can be collinear with the plasma nozzle.

According to a second aspect there is provided the device described herein for use in therapy.

According to a third aspect there is provided the device described herein for use in the treatment of skin disorders, in particular, Dermatomycosis or Onychomycosis.

Onychomycosis is a nail disease of the toes and fingers typically caused by the organisms Candida albicans, Trichophyton mentstgmphytes, Trichophyton rubrum, or Epidermpophyton floceusimi. The nails become thickened and lustreless, and debris accumulates under the free edge. Nail plates becomes separated and the nails may be destroyed. It is acknowledged that the therapy of onychomycosis is difficult and protracted. Oral therapy with antimycotics requires months of administration and must be closely monitored for side effects.

Topical compositions have long been used with the objective of treating onychomycosis. Yet these chemical based topical applications have been largely unsuccessful because the nail is a difficult barrier for anti-fungal compounds to penetrate. To be effective a topical treatment for onychomycosis should exhibit a powerful potency for pathogens. It must also be permeable through the nail barrier, and safe for patient use. There exists a need in the art for a topical application that combines these traits in high degree.

Non-thermal plasmas have long been known to exhibit biocidal properties yet none of the prior art has addressed the issue of targeting an infection under a nail and the associated permeability issues. Nor have they looked at the treatment of the pathogens that surround the infection, which is untreated, can lead to the spread of the infection or the re-infection of the digit.

Accordingly, there remains a need in the art for a topical application which can be safely applied to nails of fingers and toes, and which exhibits in combination permeability and potency for pathogens required to effectively cure, or prevent the spread of onychomycosis.

The device of the invention provides a unique means for treating onychomycosis. Advantageously, such means provides, in combination, certain characteristics, including safety, effectiveness, convenience, and freedom from toxicity, which have been unavailable heretofore. Through in vitro microbiological tests it is now found that a topical application of Plasma using the device described herein, a topical application regime can be provided to a patient to effectively penetrate the nail and kill of the bacteria causing the disease.

Without wishing to be bound by theory, it is further considered that the application of the laser to the treatment site may stimulate blood flow, while also providing high energy doses of light energy to the treated site. This can have a strong anti-fungal effect, for example. Indeed, thermal lasers may be used for treating verracae, acne and other skin issues.

### FIGURES

The present disclosure will be described in relation to the following non-limiting figures, in which:

FIG. 1 is a schematic of a device according to the present invention.

FIG. 2 depicts the use of two laser generating devices to allow for optimum spacing of the device from a surface to be treated.

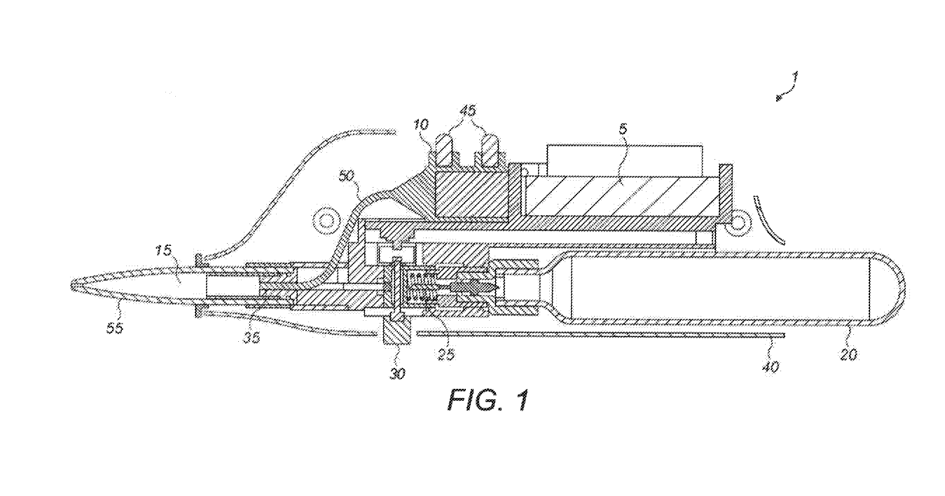

Specifically, FIG. 1 shows a combined treatment device 1. The device 1 is hand-held and incorporates a high voltage battery power pack 5. The power pack 5 is for powering the laser diode module 10 and for generating plasma in the plasma chamber 15.

The device 1 further includes a gas supply cylinder 20 containing a suitable gas at a pressure of 190 Bar. The cylinder 20 is in fluid connection with the plasma chamber 15 via a gas pressure regulator 25, operated with a manual button 30, and a plasma feed gas channel 35. The plasma chamber 15 includes electrodes (not shown) for the generation of a plasma.

The power pack 5 and the cylinder 20 are contained entirely within a housing 40.

The laser diode module 10 is controlled by laser operation buttons 45 and the laser is conducted via a laser light guide 50 to the plasma chamber 15. The laser light is aligned concentrically with the plasma outlet.

In use plasma is generated in the plasma chamber 15 and flows out of a quartz/borosilicate plasma delivery nozzle 55 onto a surface to be treated. The concentrically aligned laser is applied to the same surface to be treated.

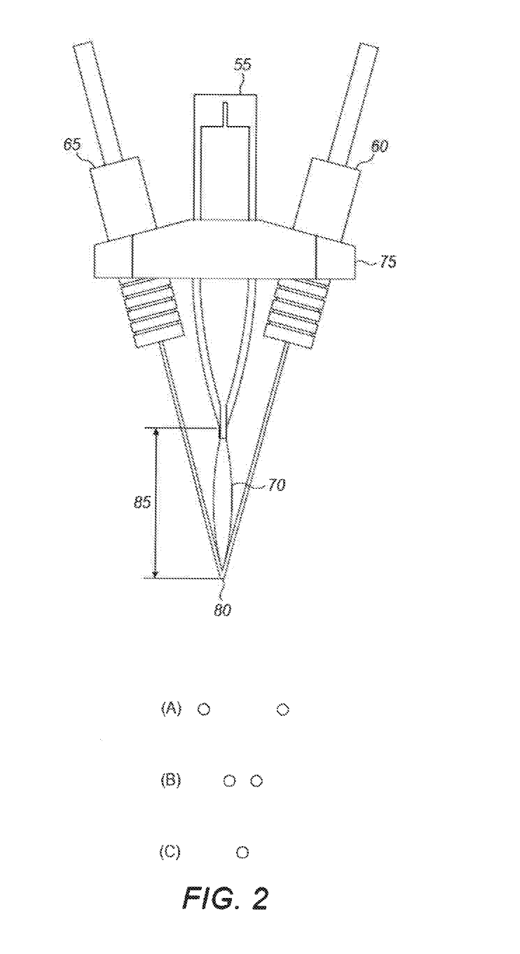

FIG. 2 shows a configuration of a plasma deli very nozzle 55 and first and second lasers 60, 65. The nozzle 55 produces a plasma plume 70 which is spaced at an optimum distance 85 from a surface to be treated 80 when the two lasers 60, 65, converge. The nozzle 55 and the lasers 60, 65 are held in the desired orientation by a clamp 75.

FIG. 2 further shows examples of the laser dots on a treatment surface. In (A) the device 1 is either too close or too far away from the treatment site. In (B) the device 1 is closer to an optimum spacing. In (C) the spacing is optimum.

### EXAMPLES

The present disclosure will now be described in relation to the following non-limiting examples.

Part. A—Importance of Archieving the Correct Distance

The objective was to find the most effective distance between the plasma nozzle outlet and the target and understand how sensitive the plasma effect is to distance.

This was done by measuring the bleaching effect on Parasure plasma indicator strips by measuring the L*a*b* colour change following a 30 minute application of different gas mix plasmas.

The following tests were carried out using the “PF4” device and experimental rig whereby a base control unit provides the required gas flow and electrical supply via an umbilical cord to a hand held unit. The hand held unit consists of concentric inner and outer barrier electrodes mounted on quartz tubes to which a high voltage is applied and between which the gas is flowed. The discharge plasma gas flows down the open quartz flow tube and into the atmosphere. The main discharge strikes across the narrow gap between the inner and outer electrodes but a secondary discharge occurs down the flow tube in to the plume formed by the flow of plasma gas mixing with the air at the end of the flow tube.

The gas flow rate used was 2.5 LPM, the power settings were 8.25 kV and the gas mixes were varied by means of two mass flow controllers. The L*a*b* colour of the strips was measured using a spectrophotometer.

Test A1—Kr/Ar mixes

Delta L* colour change of plasma strips following 30 min plasma treatment at different distances and gas concentrations are shown in the following table.

| Delta L* | 2 mm | 4 mm | 6 mm | 8 mm | 10 mm | 12 mm |
|---|---|---|---|---|---|---|
| 4% Kr/Ar | 10 | 10 | 34 | 30 | 25 | 16 |
| 1% Kr/Ar | — | 15 | 35 | 28 | 28 | — |

The data show that:
Both plasmas have a peak of effectiveness around 6 mm.
The effectiveness of both plasmas rapidly deteriorates as the distance reduces or increases around this optimum distance.

### Part.B—Beneficial Biocial Effect of Combining Plasma and Laser

The objective was to understand whether there is any beneficial synergistic effect on the treatment of onychomycosis by combining known cold laser therapy with novel plasma therapy.

All of the following tests were carried out using a Medpharm Ltd infected nail model (ChubTur®) whereby full thickness human nail samples are inoculated with spores of Trichophyton Rubrum and incubated for 14 days in a hydrated warm environment to allow the fungus to grow in to the nail. The nail samples were set in a cell apparatus and exposed to both plasma and laser treatment (Erehonia™ non-thermal laser). Plasma was applied for 1minute as we know this has a measureable but incomplete effect and the laser for 12 minutes as this is the manufacturer recommended dose for a single visit (6-8 doses recommended).

Measurements of effectiveness are derived from an ATP assay following 24 hrs incubation. In this model, the amount of luminescence measured is directly proportional to the amount of ATP present, where the level of ATP detected is an indication of the viability of T. Rubrum. Experiments are based on a sample size of 4.

The following tests were again carried out using the “PF4” device and experimental rig.

### Test B1—Combined Laser and Plasma Treatment of Infected Nails

The following table shows the percentage of remaining viable fungus following single treatments.

| | Plasma | Plasma + laser |
|---|---|---|
| Control | 100% | 100% |
| Single treatment | 25% | 17% |

The data shows that the combination of laser and plasma is beneficial and superior to plasma alone due to the higher effective kill rate (83% compared to 75%).

The foregoing examples confirm that the co-treatment of a surface with laser and plasma has a beneficial therapeutic effect. Moreover, since the efficacy of the treatment is extremely dependent on the spacing of the plasma source from the treatment site, the use of laser-aided spacing of the plasma device from a treatment surface can be surprisingly beneficial.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

1. A plasma-generation device for applying plasma to a human body, the device comprising:
a reservoir containing a gas,
a plasma zone in fluid connection with the reservoir,
means for generating a plasma by electrical discharge in the plasma zone, and a first laser generating device,
wherein the plasma zone has an outlet for dispensing plasma and wherein the first laser generating device is arranged to generate a laser for simultaneous plasma and laser treatment of a surface.

2. The device of claim 1, wherein the outlet is a nozzle.

3. The device according to claim 2, wherein the first laser generating device is arranged to generate a laser that is collinear with a longitudinal axis of the nozzle.

4. The device according to claim 1, wherein the first laser generating device is aligned with the outlet for simultaneous plasma and laser treatment of a surface.

5. The device according to claim 1, further comprising a second laser generating device, and wherein the first and second laser generating devices are arranged to generate lasers which intersect at a predefined spacing from the outlet.

6. The device according to claim 5, wherein the first laser generating device and the second laser generating device each operate a laser having an operating frequency in the range of from 10 to 635 nm.

7. The device according to claim 1, wherein the means for generating a plasma comprises a power supply, and first and second electrodes, and wherein, in use, the plasma zone is formed between the first and second electrodes and wherein a flow of gas from the reservoir through the plasma zone provides a flow of plasma to contact a surface of a human body.

8. The device according to claim 1, wherein the device is hand-held.

9. The device according to claim 8, wherein the power supply comprises a battery integrated into the hand-held device.

10. The device according to claim 1, wherein the means for generating a plasma operates at a voltage of from 2-15V.

11. The device according to claim 1, wherein the device is a hair straightener, a toothbrush, foot-spa or a hair-brush.

12. The device according to claim 1, wherein the gas comprises Helium, Argon, Neon, Krypton, or Hydrogen, or mixtures of two or more thereof.

13. A method for the treatment of a surface using a plasma-generation device, the device comprising a reservoir containing a gas a plasma zone in fluid connection with the reservoir, means for generating a plasma by electrical discharge in the plasma zone, and a first laser generating device, wherein the plasma zone has an outlet for dispensing plasma and wherein the first laser generating device is arranged to generate a laser for simultaneous plasma and laser treatment of the surface; the method comprising:
flowing gas from the reservoir to the plasma zone and generating a plasma by electrical discharge in the plasma zone, and
simultaneously contacting a surface with the plasma and laser radiation from the laser.

14. The method according to claim 13, wherein the gas is supplied through the means for generating a plasma at a flow rate of less than 5 l/min.

15. The method according to claim 13, wherein the gas is flowed to the plasma zone at a pressure of from 1 to 2 bar.

16. A device according to claim 1, for use in therapy.

17. A device according to claim 1, for use in the treatment of skin and nail disorders.

18. The device according to claim 17, wherein the skin disorder is Dermatomycosis or Onychomycosis,

19. The device according to claim 6, wherein operating frequency is in the range of from 405 to 635 nm.
